(19) United States
(12) Patent Application Publication
(10) Pub. No.: US 2026/0315159 A1
(43) Pub. Date: Oct. 15, 2026

(54) SYSTEM AND METHOD FOR REAL-TIME PROJECTION OF 3D MEDICAL IMAGE INFORMATION ONTO A PATIENT VIA TRACKING OF SKIN FIDUCIALS ON THE PATIENT

(71) Applicant: ILLUMINANT SURGICAL, INC., Palo Alto, CA (US)

(72) Inventors: James Benjamin Hu, Long Beach, CA (US); Jhoneldrick Saleh Millares, Los Angeles, CA (US); Enoch Omale, Los Angeles, CA (US); Nicholas Grant Shelton, Los Angeles, CA (US); Brian Minh Nguyen, Culver City, CA (US); Alexa Alisa Nazarian, Glendale, CA (US); Harry Dung Trung Nguyen, Los Angeles, CA (US); Sevryn Aubrey Gott, New York, NY (US); Julian Valentin Javier, New York, NY (US); Lillian Yihui Mo, Palo Alto, CA (US)

(21) Appl. No.: 19/576,796

(22) Filed: Mar. 24, 2026

Related U.S. Application Data
(60) Provisional application No. 63/776,735, filed on Mar. 24, 2025.
(60) Provisional application No. 63/816,234, filed on Jun. 2, 2025.

(51) Int. Cl. A61B 34/20; A61B 90/00

(52) U.S. Cl. CPC A61B 34/20; A61B 90/37; A61B 90/39; A61B 2034/2055; A61B 2090/3762; A61B 2090/3908

(57) ABSTRACT

One variation of a surgical tracking system includes: a set of fiducials configured to affix to skin on a region of a body of a patient during a medical procedure, each fiducial including a retroreflective spherical section; and a projection system including an optical sensor, a projector, and a computer system. The computer system is configured to: derive a set of spatial coordinates of fiducials on the body based on features extracted from an image captured by the optical sensor; detect a constellation of fiducials in a 3D-medical image depicting the region; calculate a transform between the 3D-medical image and the image by aligning the constellation of fiducials to the set of spatial coordinates; and, based on the set of spatial coordinates and the transform, trigger the projector to project an overlay of the 3D-medical image onto the region of the body during the medical procedure.

### CROSS-REFERENCE

This Application claims the benefit of U.S. Provisional Application No. 63/816,234, filed on 2 Jun. 2025, and U.S. Provisional Application No. 63/776,735, filed on 24 Mar. 2025, each of which is incorporated in its entirety by this reference.

### TECHNICAL FIELD

This invention relates generally to the field of image-guided surgery and, more specifically, to a new and useful system and method for projection of medical image information onto a human body via tracking of IR-detectable, skin-adhesive fiducials in the field of image-guided surgery.

### BRIEF DESCRIPTION OF THE FIGURES

FIG. 1 is a schematic representation of a surgical tracking system;

FIG. 2 is a schematic representation of one variation of the surgical tracking system;

FIGS. 3A and 3B are flowchart representation of a method; and

FIG. 4 is a flowchart representation of one variation of the method.

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### 1. Surgical Tracking System: Skin Fiducial

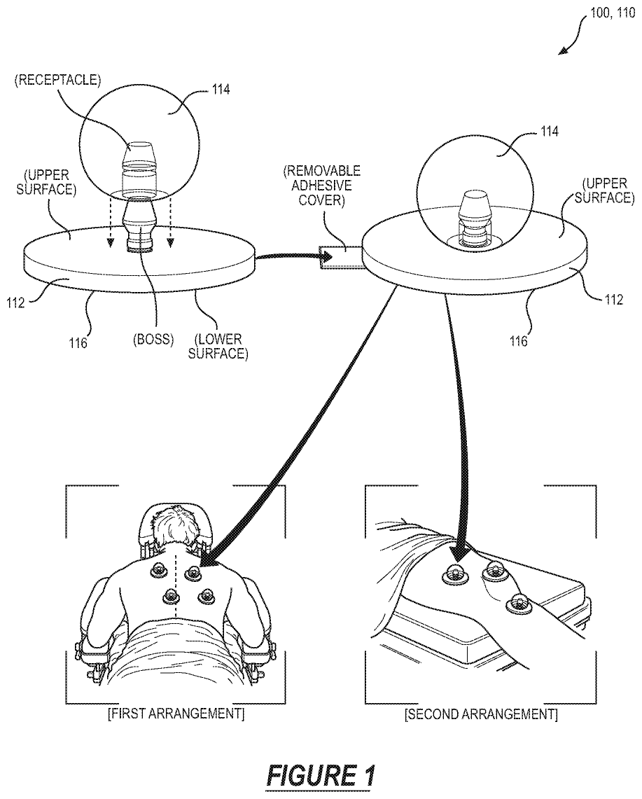

As shown in FIGS. 1, 2, 3A, 3B, and 4, a surgical tracking system 100 includes a first skin fiducial 110, in a set of skin fiducials 110, configured to transiently affix to skin of a human patient occupying an operating space during a medical procedure, the first skin fiducial 110 including: a base section 112; a spherical section 114; and an adhesive layer 116.

The base section 112 defines: an upper surface configured to be non-reflective in an infrared spectrum to reduce interference with optical tracking; a bottom surface opposite the upper surface and configured to seat flush against skin of the human patient; and a boss extending upward from the upper surface.

The spherical section 114: is formed of a retroreflective material configured to reflect infrared light for detection of the spherical section 114 in infrared images; and is mechanically coupled to the base section 112. The spherical section 114 defines: an upper section defining a spherical geometry; a truncated base defining a base surface configured to mate with the upper surface of the base section 112; and a receptacle extending inward from the truncated base and configured to receive and retain the boss of the base section 112 to mechanically couple the spherical section 114 to the base section 112.

The adhesive layer 116 is: applied to the bottom surface of the base section 112; formed of a pressure-sensitive adhesive material; and configured to transiently affix the base section 112 to skin of the human patient during the medical procedure.

### 1.1 Surgical Tracking System: Skin Fiducials+Sensors+Projector

As shown in FIGS. 1, 2, 3A, and 3B, one variation of the surgical tracking system 100 includes: a set of skin fiducials 110 configured to transiently affix to skin of a human patient occupying an operating space during a medical procedure, each skin fiducial 110 including a retroreflective spherical section 114 configured to reflect infrared light; and a projection system 120 including an optical tracking sensor 122, a projector 130, and a computer system 150.

The optical tracking sensor 122 is configured to capture images of a region of a body of the human patient, the region of the body including the set of skin fiducials 110. The projector 130 is configured to project visual overlays of three-dimensional medical images of the human patient onto the region of the body. The computer system 150 is configured to: detect the set of skin fiducials 110 in an image captured by the optical tracking sensor 122; derive a set of spatial coordinates of the set of skin fiducials 110 on the region of the body relative the projector 130 based on features extracted from the image; access a three-dimensional medical image of the region of the body captured by an intraoperative scanner arranged in the operating space, the three-dimensional medical image depicting a set of anatomical features of the human patient corresponding to the region of the body; detect a constellation of skin fiducials 110 in the three-dimensional medical image, the constellation of skin fiducials 110 representing the set of skin fiducials 110 on the body; calculate a transform between the three-dimensional medical image and the image by aligning the constellation of skin fiducials 110 in the three-dimensional medical image to the set of spatial coordinates to locate features of the three-dimensional medical image in the operating space; generate a visual overlay based on the set of spatial coordinates and the transform; and trigger the projector 130 to project the visual overlay onto the region of the body during the medical procedure.

In one variation, the projection system 120 further includes: a mobile cart 140 transiently arranged within the operating space and housing the computer system 150; and a housing 142 mounted to the mobile cart 140. In this variation, the optical tracking sensor 122 and the projector 130 are rigidly integrated within the housing 142.

### 1.1.1 Surgical Tracking System: Infrared Sensor+Depth Sensor

In one variation, the optical tracking sensor 122 of the projection system 120 is an infrared sensor 124 configured to capture infrared images of the region of the body. In this variation, the projection system 120 further includes a depth sensor 126 configured to capture depth images of the region of the body. The computer system 150 is configured to: detect the set of skin fiducials 110 in an infrared image captured by the infrared sensor 124; derive the set of spatial coordinates of the set of skin fiducials 110 on the region of the body relative the projector 130 based on features extracted from the infrared image; calculate the transform between the three-dimensional medical image and the infrared image by aligning the constellation of skin fiducials 110 in the three-dimensional medical image to the set of spatial coordinates to locate features of the three-dimensional medical image in the operating space; generate a surface model of the region of the body based on depth data captured by the depth sensor 126; and generate the visual overlay based on the set of spatial coordinates, the transform, the surface model, and the visual overlay configured to conform to curvature of the surface of the region of the body during projection onto the region of the body.

### 1.1.2 Surgical Tracking System: RGB+Depth Sensor

In one variation, the optical tracking sensor 122 of the projection system 120 is a color and depth camera configured to capture depth data and RGB images of the region of the body. In this variation, the projection system 120 further includes a depth sensor 126 configured to capture depth images of the region of the body. The computer system 150 is configured to: generate a surface model of the region of the body based on depth data captured by the depth sensor 126; and generate the visual overlay based on the set of spatial coordinates, the transform, and the surface model, the visual overlay configured to conform to curvature of the surface of the region of the body during projection onto the region of the body.

### 2. Method

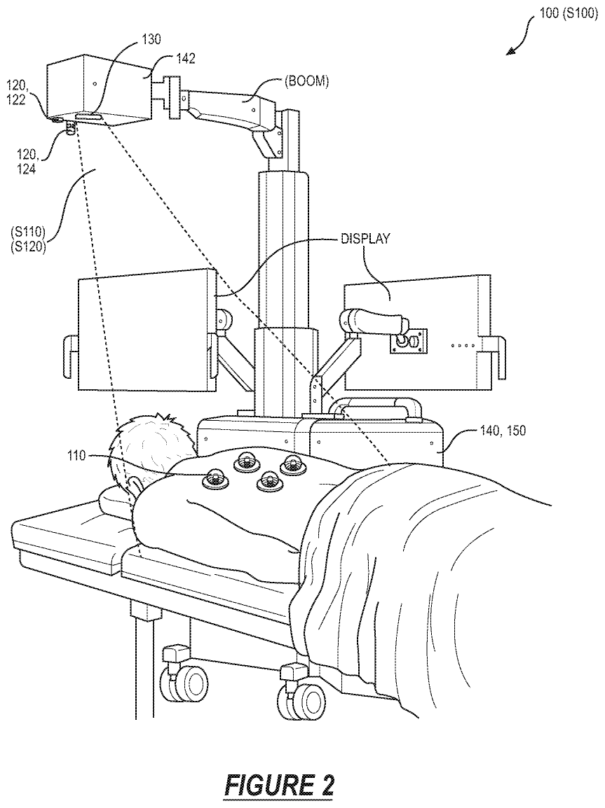

As shown in FIGS. 2, 3A, 3B, and 4, a method S100 includes: accessing an infrared image of a region of a body and captured by an infrared sensor 124 arranged at a first location in an operating space in Block S110; and detecting a set of skin fiducials 110 arranged in first arrangement on the body in the infrared image in Block S112, each skin fiducial 110, in the set of skin fiducials 110, including a base section 112, an adhesive layer 116 applied to the base section 112 and configured to transiently affix the base section 112 to skin of the body, and a spherical section 114 coupled to the base section 112 and including a retroreflective surface; for each skin fiducial 110, in the set of skin fiducials 110, calculating a set of location coordinates in the operating space based on features extracted from the infrared image in Block S114; accessing a three-dimensional medical image of the region of the body, the three-dimensional medical image captured by an intraoperative scanner arranged at a second location in the operating space in Block S120; detecting a constellation of skin fiducials 110 in the three-dimensional medical image, the constellation of skin fiducials 110 representing the set of skin fiducials 110 on the body in Block S122; calculating a transform between the three-dimensional medical image and the infrared image by aligning the set of skin fiducials 110 in the three-dimensional medical image to the set of location coordinates of the set of skin fiducials 110 in the operating space to locate features of the three-dimensional medical image in the operating space; accessing a location of a projector 130 in the operating space in Block S130; based on the location of the projector 130, the set of location coordinates, and the transform, generating a visual overlay of the three-dimensional medical image configured for projection onto the region of the body of the patient, the constellation of skin fiducials 110 depicted in the three-dimensional medical image aligned to the set of skin fiducials 110 applied to region of the body in Block S140; and triggering the projector 130 to project the visual overlay onto the region of the body in the operating space in Block S142.

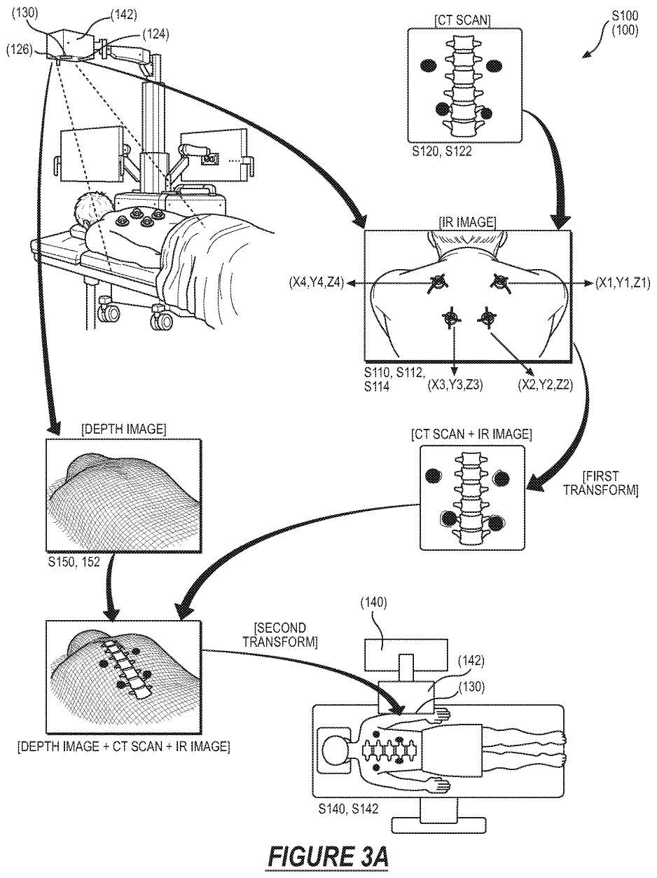

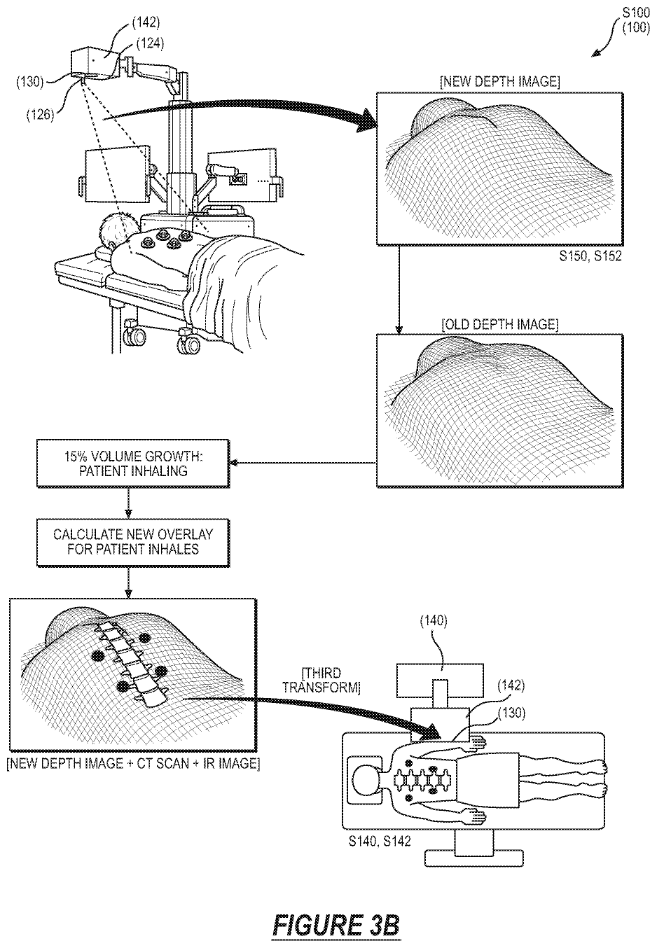

In one variation, as shown in FIGS. 3A and 3B, the method S100 further includes: accessing a depth image depicting the region of the body and captured by a depth sensor 126 arranged in the operating space in Block S150; and defining a three-dimensional mesh representing a surface of the region of the body based on features extracted from the depth image in Block S152. In this variation, the method S100 includes generating the visual overlay of the three-dimensional medical image based on the location of the projector 130, the set of location coordinates, the transform, and the three-dimensional mesh, the visual overlay configured to conform to curvature of the surface of the region of the body during projection onto the region of the body.

### 2.1 Method: Skin Fiducial Arrangement Verification

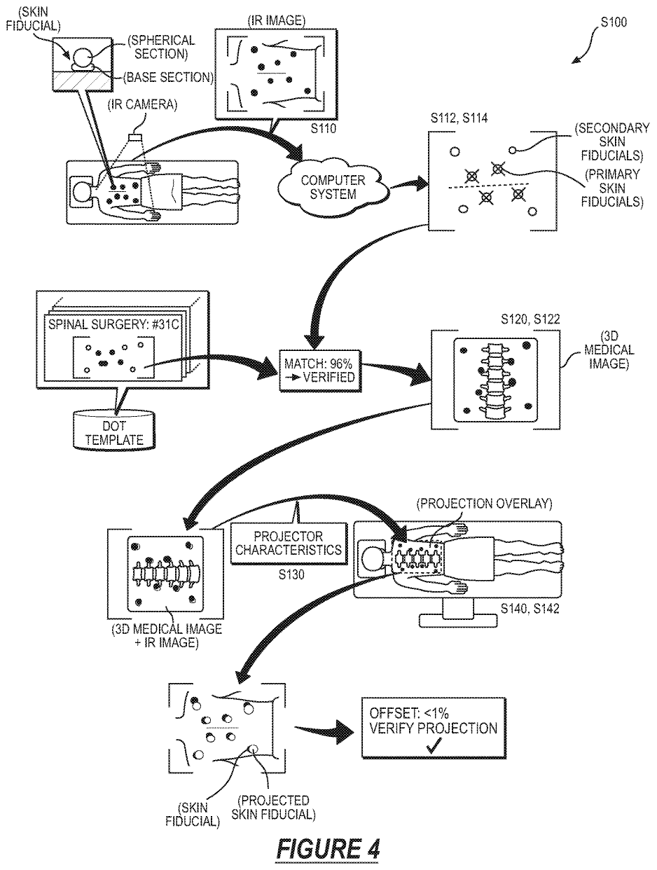

As shown in FIG. 4, one variation of the method S100 includes: accessing an IR image of a region of a human body and captured by an IR camera arranged at a first location in an operating space; and detecting a set of skin fiducials 110-arranged in first arrangement-on the human body in the IR image, each skin fiducial 110, in the set of skin fiducials 110, including: a base section 112; an adhesive layer 116 applied to base section 112 and configured to transiently affix the base section 112 of the skin fiducial 110 to skin of a patient; and a spherical section 114 coupled to the base section 112 and including a retroreflective surface detectable via an IR camera. The method S100 further includes: for each skin fiducial 110, in the set of skin fiducials 110, calculating a set of location coordinates in the operating space; receiving confirmation of a medical procedure of a first procedure type for the patient; accessing a first fiducial template, in a set of fiducial templates, corresponding to the first procedure type in Block S160; characterizing a difference between the first arrangement and a target arrangement of skin fiducials 110 defined by the first fiducial template in Block S162; and, in response to the difference falling below a threshold difference, verifying the first arrangement for the medical procedure in block S164.

The method S100 further includes: accessing a 3D medical image (e.g., X-ray or CT scan) of the region of the patient's body and captured by an intraoperative scanner at a second location in the operating space in Block S120; detecting a constellation of skin fiducials 110—representing the set of skin fiducials 110 on the patient's body—in the 3D medical image in Block S122; calculating a transform between the 3D medical image and the IR image by aligning the set of skin fiducials 110 in the 3D medical image to the set of location coordinates—derived from the IR image—of the set of skin fiducials 110 in the operating space to locate features of the 3D medical image in the operating space in Block S130; accessing a location of the projector 130 in the operating space; based on the location of the projector 130, the set of location coordinates, and the transform, generating a projection overlay of the 3D medical image configured for projection, via the projector 130, onto the patient's body, the constellation of skin fiducials 110 depicted in the 3D medical image aligned to the set of skin fiducials 110 on the patient's body in Block S140; and triggering the projector 130 to project the projection overlay onto the human body in the operating space in Block S142.

In one variation, the method S100 includes, in response to the difference exceeding the threshold difference: flagging the first arrangement as a setup error in Block S166; generating a notification indicating the setup error and including a prompt to readjust placement of the set of skin fiducials 110 on the patient's skin; and transmitting the prompt to a medical professional affiliated with the medical procedure in Block S168.

### 3. Applications

Generally, the method S100 can be executed by a computer system 150 (e.g., a computer network, a remote computer system 150, a local or remote server) —interfacing with one or more optical tracking sensors 122 arranged within an operating space (e.g., an operating room)—to: detect a set of retroreflective skin fiducials 110 (i.e., spherical, retroreflective fiducials)—arranged on skin of a region of a patient's body in preparation for a medical procedure for this patient—in an IR image captured by an IR sensor arranged in the operating space; detect these skin fiducials 110 in a 3D medical image (e.g., an X-ray, a CT scan)—depicting the region of the patient's body and/or relevant body features associated with the medical procedur—captured by an intraoperative scanner transiently installed in the operating space; map the 3D medical image to the patient's body in the operating space based on detected locations of the set of retroreflective skin fiducials 110 in the IR image and the 3D medical image; access a location and a field of view of a projector 130 installed in the operating space; based on the location and the field of view of the projector 130, generate a projection overlay of the region of the patient's body depicted in the 3D medical image configured to align to the patient's body when projected via the projector 130; and trigger the projector 130 to project the projection overlay onto the region of the patient's body.

Therefore, a medical professional (e.g., a surgeon) performing the medical procedure for this patient may view the projection overlay on the patient's body during execution of the medical procedure. By projecting this information onto the patient's body, the computer system 150 can enable the medical professional to view all relevant information on the patient's body-such as without looking away to a separate screen including this information-during execution of the medical procedure, thereby reducing the cognitive load of the medical professional. Furthermore, the computer system 150 can enable the medical professional to execute various procedures—such as cutting into the patient's body and/or drilling into a bone—at a precise location defined for a medical procedure type on a first attempt, rather than requiring the medical professional to initially execute a test to identify precise locations within the patient's body, thereby enabling non-invasive and accurate patient access.

Generally, the computer system 150 can detect skin fiducials 110 —arranged in a particular pattern or arrangement on the patient's body—in IR images, RGB images, and/or 3D medical images (e.g., X-ray, CT scan). For example, each skin fiducial 110 can include: a base configured to transiently seat (approximately flush) against the patient's skin; an (skin-safe) adhesive layer 116 applied to a bottom surface of the base and configured to transiently affix the bottom surface of the base to the patient's skin; and a spherical section 114 —coupled to the plastic base—defining a spherical geometry and including a retroreflective surface. Therefore, the computer system 150 can accurately detect the spherical section 114 of each skin fiducial 110 in images due to the spherical geometry enabling detection from any viewing angle while maintaining a uniform geometry, and the retroreflective surface enabling tracking of the skin fiducial 110 via IR tracking methods.

Therefore, the computer system 150 can leverage detection of the set of skin fiducials 110 on the patient's body—detected in both the IR image (and/or RGB image) and the 3D medical image (e.g., CT scan, X-ray)—to generate and project a projection overlay of the 3D medical image onto the patient's body such that features depicted in the 3D medical image align with these features of the patient's body.

Furthermore, in one implementation, the computer system 150 can leverage detection of the set of skin fiducials 110 in the IR image and/or the 3D medical image to confirm a setup prior to initiation of a medical procedure for the patient. For example, the computer system 150 can: access an IR image—captured by one or more IR cameras arranged in the operating space—of a region of a patient's body and depicting the set of skin fiducials 110; detect the set of skin fiducials 110 in the IR image; interpret a set of location coordinates of each skin fiducial 110, in the set of skin fiducials 110, in the IR image; retrieve a fiducial template—corresponding to a medical procedure type (e.g., lumbar surgery, knee surgery, thoracic surgery, spinal surgery, an epidural injection, vertebral augmentation) of the medical procedure and defining a target arrangement of skin fiducials 110—from a template database; interpret a first arrangement of the set of skin fiducials 110 on the patient's body based on the set of location coordinates of each skin fiducial 110 in the set of skin fiducials 110; and characterize a difference between the first arrangement of the set of skin fiducials 110 on the patient's body and the target arrangement of skin fiducials 110 defined by the fiducial template for the medical procedure type. In response to the difference falling below a threshold difference, the computer system 150 can verify the setup for this medical procedure and/or notify the medical professional of verification of the setup. Alternatively, in response to the difference exceeding the threshold difference, the computer system 150 can notify the medical professional of failure to verify the setup and/or prompt the medical professional to adjust placement of the set of skin fiducials 110 on the patient's body and re-confirm the setup accordingly.

In this implementation, the computer system 150 can, therefore, limit resources dedicated by a surgeon and/or other medical professional performing the medical procedure by ensuring proper setup of the set of skin fiducials 110 prior to initiation of the medical procedure. Furthermore, the computer system 150 can promote placement of the set of skin fiducials 110 in particular regions of the body predicted to enable better detection of skin fiducials 110 and limit interference with the medical procedure.

### 3.1 Applications: Projection System

In one implementation, the surgical tracking system 100 includes: a set of adhesive skin fiducials 110 arranged on skin of a region of a patient's body, each skin fiducial 110 functioning as a temporary marker configured to enable accurate registration between physical anatomy of the patient during a medical procedure and medical imaging data captured for the patient; an infrared sensor 124 (e.g., an infrared (IR) tracking camera) arranged within an operating space (e.g., an operating room) and configured to capture infrared images depicting retroreflective spherical sections 114 of the set of skin fiducials 110 and, thereby, enable the computer system 150 to interpret spatial coordinates of each skin fiducial 110 in (near) real time; a depth sensor 126 configured to capture three-dimensional surface data of the region of the patient's body and, thereby, enable the computer system 150 to derive a surface model representing curvature and/or contour of a surface of the region of the patient's body; and a projector 130 —arranged within the operating space and defining a field of view oriented toward the region of the patient's body—configured to project a visual overlay derived from a three-dimensional medical image (e.g., CT, X-ray) onto the region of the patient's body based on alignment between detected skin fiducials 110 in the operating space and corresponding fiducials detected in the three-dimensional medical image.

The computer system 150 can thus compute a spatial transform that aligns medical imaging data (captured just before or in real time) with the patient's physical anatomy. The projector 130, positioned at a known location relative to the sensors, then projects a calculated visual overlay—including anatomical structures, planned trajectories, and/or cross-sectional image slices—directly onto the patient's body.

Therefore, the set of skin fiducials 110 function as stable reference markers that connect imaging data, surface mapping, and projected visualization into a unified, real-time image-guidance platform for a medical professional conducting a medical procedure, enabling the medical professional (e.g., a surgeon) to view anatomical information directly on the patient's body during execution of the medical procedure. Furthermore, by geometrically calibrating the depth sensor 126, infrared sensor 124 (or “tracking sensor”), and projector 130 within a shared coordinate framework, the computer system 150 can continuously update the projection to maintain alignment during the medical procedure, regardless of whether the patient or equipment shifts slightly during the medical procedure.

In particular, by integrating the infrared sensor 124, the depth sensor 126, and the projector 130 within a unified spatial framework, the computer system 150 can continuously align projected imaging data to the patient's anatomy without requiring the medical professional to divert attention to a remote display. Furthermore, by maintaining alignment between the projected overlay and the physical anatomy in (near) real time, the projection system 120 can enable the medical professional to execute procedures—such as incisions, drilling, implant placement, needle insertion, etc.—at precise anatomical locations defined by the three-dimensional medical image, thereby improving targeting accuracy and workflow efficiency.

Generally, the computer system 150 leverages structural characteristics of each skin fiducial 110 to support multimodal detection and tracking of the set of skin fiducials 110. For example, each skin fiducial 110 can include: a rigid base configured to seat substantially flush against the patient's skin; a medical-grade adhesive layer 116 applied to a bottom surface of the base and configured to transiently affix the base to the patient's skin; and a spherical section 114 mechanically coupled to the base and defining a substantially spherical exterior geometry formed of a retroreflective and radiopaque material. The spherical geometry can enable consistent detection from multiple viewing angles by the infrared sensor 124, while the retroreflective material can facilitate high-contrast detection in IR images. Additionally, the radiopaque characteristic of the spherical section 114 can enable detection of the skin fiducial 110 within a three-dimensional medical image, thereby providing a common reference feature across optical and radiographic sensing modalities.

Therefore, the computer system 150 can detect the set of skin fiducials 110 in both an image captured by the infrared sensor 124 (or other optical tracking sensor types) and in a three-dimensional medical image acquired via an intraoperative scanner (e.g., a CT scanner), and compute a spatial transform aligning the three-dimensional medical image to the patient's physical anatomy in the operating space. The depth sensor 126 can further capture surface geometry of the region of the patient's body and enable distortion compensation when generating a projection overlay for the projector 130. Based on detected positions of the set of skin fiducials 110 and the surface model, the computer system 150 can generate a projection overlay configured to align to the region of the patient's body when projected. The projector 130 can then project the projection overlay directly onto the patient's body such that anatomical structures depicted in the medical image correspond to their physical locations.

### 4. Skin Fiducials

Generally, the surgical tracking system 100 includes an array of skin fiducials 110 (or “skin dots”) transiently affixed to a human body in an image (e.g., a 2D or 3D image) of the human body and detectable by the computer system 150 in images captured by one or more optical tracking sensors 122, such as including an infrared sensor 124 and/or a depth sensor 126. In particular, each skin fiducial 110 defines an adhesive fiducial configured to transiently affix to skin on the human body via an adhesive layer 116 of the skin fiducial 110.

In one implementation, each skin fiducial 110 includes: a base section 112 including an adhesive layer 116 applied to a bottom surface of the base section 112 and configured to stick to skin on the human body; and a spherical section 114 —defining a spherical geometry—coupled to the base section 112 and defining a retroreflective surface configured to enable detection of the skin fiducial 110 in images via application of infrared light to the skin fiducial 110. For example, each skin fiducial 110 can include: a plastic base configured to transiently seat (approximately flush) against the patient's skin; a skin-safe adhesive layer 116 applied to a bottom surface of the base and configured to transiently affix the bottom surface of the plastic base to the patient's skin; and a spherical section 114 —coupled to the plastic base—defining a spherical geometry of a fixed diameter (e.g., 11.4 millimeters) and including a radiopaque and retroreflective surface.

In particular, in one example, each skin fiducial 110 includes: the base section 112 formed of a polycarbonate material; the spherical section 114 formed of a polymethyl methacrylate material including a retroreflective coating including glass microbeads; and the adhesive layer 116 formed of a pressure-sensitive acrylic material.

In this implementation, by including the spherical section 114 defining the spherical geometry, the skin fiducial 110 can be configured to enable detection of the skin fiducial 110 from any viewing angle-defined by the optical tracking sensor 122 capturing images of the skin fiducial 110 on the human body —while maintaining a fixed 2D geometry depicted in these images. Furthermore, by including a retroreflective surface on the spherical section 114, the skin fiducial 110 can be configured to enable tracking of the skin fiducial 110 via infrared sensing by efficiently reflecting infrared light—emitted by an infrared light camera —back toward the infrared light camera. Therefore, the computer system 150 can detect the skin fiducial 110 in an image—such as in an IR image, an RGB image, and/or a 2D or 3D medical image—and track the skin fiducial 110 in (near) real time via infrared-light tracking.

Each skin fiducial 110 thus functions as a temporary marker configured to enable accurate registration between physical anatomy of the patient and three-dimensional medical images captured for the patient. For example, the set of skin fiducials 110 can be detected in images captured by an infrared sensor 124 and/or by a computed tomography (or “CT”) scanner or other medical imaging systems. The computer system 150 can leverage detected locations of the set of skin fiducials 110—extracted from both medical imaging data and/or optical data captured by the optical tracking sensors 122 of the projection system 120—to compute spatial transformations that align three-dimensional medical images with the patient's body in the operating space.

### 4.1 Base Section

In one implementation, the base section 112 includes a rigid body formed of a biocompatible material and defines an upper surface and a bottom surface opposite the upper surface. The bottom surface is configured to seat substantially flush against skin of the human patient in order to limit positional error caused by deformation of the skin during the medical procedure. The upper surface is configured to be non-reflective in an infrared spectrum such that infrared light emitted by an infrared sensor 124 does not produce unwanted reflections that could interfere with optical detection of the set of skin fiducials 110.

For example, the base section 112 can include a polymer material suitable for injection molding, such as polycarbonate, acrylonitrile butadiene styrene (ABS), polypropylene, nylon, or another medical-grade thermoplastic material. The base section 112 can further include a surface treatment or pigmentation configured to reduce reflectivity in an infrared spectrum so that the base section 112 does not produce reflections that could interfere with detection of the spherical section 114 by an infrared sensor 124.

### 4.2 Spherical Section

Generally, the spherical section 114 is mechanically coupled to the base section 112 and defines a substantially spherical exterior geometry configured to appear as a substantially circular feature in images captured by the optical tracking sensor 122—such as including an infrared sensor 124, a depth sensor 126, and/or an RGB camera—across a range of viewing angles. By maintaining this spherical geometry, the spherical section 114 enables consistent optical detection in images captured by the optical tracking sensor 122 due to uniformity of the reflective surface, regardless of orientation relative to the optical tracking sensor 122.

Furthermore, the spherical section 114 can be configured to exhibit a diameter configured to balance detectability in optical images and medical imaging data while minimizing interference with medical instruments and access to the surgical field by the medical professional. In particular, in one implementation, the spherical section 114 defines a diameter configured to support detection across multiple sensing modalities. For example, the spherical section 114 can define a diameter configured to occupy multiple pixels in infrared images captured by an infrared sensor 124(s) of the projection system 120, thereby enabling precise calculation of a center location of the spherical section 114 in these infrared images. Additionally or alternatively, the spherical section 114 can define a diameter configured to occupy multiple voxels in three-dimensional medical images—such as including X-ray and/or CT images—thereby enabling accurate localization of the spherical section 114 within these three-dimensional medical images.

In one implementation, the spherical section 114 includes a material configured to reflect incident infrared illumination and to produce a high-contrast feature in infrared images captured by an infrared sensor 124. For example, the spherical section 114 can include a polymer sphere formed of acrylic, polymethyl methacrylate (PMMA), polycarbonate, or another optical-grade polymer. The spherical section 114 can further include a retroreflective coating or a retroreflective layer including glass microbeads embedded within a reflective matrix. The retroreflective layer can reflect incident infrared light back toward the infrared sensor 124 such that the spherical section 114 appears as a bright circular region in infrared images.

In one variation, the spherical section 114 further includes a radiopaque material configured to enable detection of the spherical section 114 within a three-dimensional medical image captured by an imaging scanner. In particular, the spherical section 114 can include and/or incorporate a radiopaque material, such that the spherical section 114 appears as a high-contrast feature within a three-dimensional medical image. For example, the spherical section 114 can include a polymer matrix including radiopaque fillers such as barium sulfate, tungsten powder, or other radiopaque compounds configured to produce contrast within computed tomography images.

The spherical section 114 further defines: a truncated base including a base surface configured to mate with the upper surface of the base section 112; and a receptacle extending inward from the truncated base. In particular, the truncated base: defines a substantially planar surface configured to seat against the upper surface of the base section 112; and is configured to stabilize the spherical section 114 relative the base section 112 during movement of the human patient. Furthermore, the base section 112 includes a boss extending upward from

the upper surface, and the receptacle of the spherical section 114 is configured to receive and retain the boss in order to mechanically couple the spherical section 114 to the base section 112. By incorporating the boss and the receptacle in the base section 112 and the spherical section 114, respectively, the base section 112 and the spherical section 114 can maintain a fixed orientation and position of the spherical section 114 relative to the base section 112 during handling and wearing of the skin fiducial 110 on the patient's body.

### 4.3 Adhesive Layer

The adhesive layer 116 applied to the bottom surface of the base section 112 includes a medical-grade pressure-sensitive adhesive configured to transiently affix the base section 112 to the skin of the human patient during the medical procedure. The adhesive layer 116 can maintain the base section 112 in contact with the skin while enabling removal of the skin fiducial 110 following completion of the medical procedure.

In one example, the adhesive layer 116 can include an acrylic pressure-sensitive adhesive, a silicone-based adhesive, or another biocompatible adhesive formulation commonly used in medical tapes and disposable medical sensors. In one example, the adhesive layer 116 can include an acrylic adhesive applied to a flexible carrier film such as polyethylene terephthalate (PET) or polyurethane and covered by a removable release liner prior to use. These adhesive materials can maintain sufficient adhesion to hold the skin fiducial 110 in place on the patient's skin during operation of the medical projection system 120 while allowing removal of the skin fiducial 110 following completion of the medical procedure without causing damage to the patient's skin.

### 4.4 Arrangement+Medical Procedure

Generally, the surgical tracking system 100 can include multiple skin fiducials 110 arranged in a particular arrangement on the patient's body.

In one implementation, the set of skin fiducials 110 can include a first skin fiducial 110, in the set of skin fiducials 110, configured to transiently affix to skin of the human patient during the medical procedure, the first skin fiducial 110 including a first base section 112 defining: an upper surface configured to be non-reflective in an infrared spectrum to reduce interference with IR optical tracking; a bottom surface opposite the upper surface and configured to seat flush against skin of the human patient; and a boss extending upward from the upper surface. The first skin fiducial 110 further includes a first spherical section 114: formed of a retroreflective material configured to reflect infrared light; mechanically coupled to the first base section 112; and defining an upper section defining the spherical geometry, a truncated base defining a base surface configured to mate with the upper surface of the second base section 112, and a receptacle extending inward from the truncated base and configured to receive and retain the boss of the second base section 112 to mechanically couple the second spherical section 114 to the second base section 112. The first skin fiducial 110 further includes a first adhesive layer 116: applied to the bottom surface of the first base section 112; formed of the pressure-sensitive adhesive material; and configured to transiently affix the first base section 112 to skin of the human patient during the medical procedure. Furthermore, the set of skin fiducials 110 can include a second skin fiducial 110 including: a second base section 112 substantially identical the first base section 112 of the first skin fiducial 110; a second spherical section 114—mechanically —coupled to the second base section 112—substantially identical the first spherical section 114 of the first skin fiducial 110; and a second adhesive layer 116—applied to the bottom surface of the second base section 112—substantially identical the first adhesive layer 116 of the first skin fiducial 110. Furthermore, the set of skin fiducials 110 can include a third skin fiducial 110 including: a third base section 112 substantially identical the first base section 112 of the first skin fiducial 110; a third spherical section 114—mechanically coupled to the third base section 112 substantially identical the first spherical section 114 of the first skin fiducial 110; and a third adhesive layer 116—applied to the bottom surface of the third base section 112—substantially identical the first adhesive layer 116 of the first skin fiducial 110.

In this implementation, the first skin fiducial 110, the second skin fiducial 110, and the third skin fiducial 110 are configured to transiently affix to skin of the human patient in a target arrangement defined for the medical procedure, such that the set of skin fiducials 110 enable accurate location and projection of relevant anatomical features without interfering with execution of the medical procedure.

In one example, a first set of skin fiducials 110—such as including a first, second, and third skin fiducial 110—can be transiently affixed to the skin of a first human patient in a first target arrangement defined for a first medical procedure associated with a first set of anatomical structures. A second set of skin fiducials 110—such as including a fourth, fifth, and sixth skin fiducial 110—can be transiently affixed to the skin of a second human patient in a second target arrangement defined for a second medical procedure associated with a second set of anatomical structures. In particular, in one example, the first set of skin fiducials 110—including the first skin fiducial 110, the second skin fiducial 110, and the third skin fiducial 110—are arranged in the first target arrangement on skin of a back of the first human patient, the first target arrangement defined for a spinal surgery and defining locations of the set of skin fiducials 110 relative to vertebrae of a spine of the human patient. The second set of skin fiducials 110—including the fourth skin fiducial 110, the fifth skin fiducial 110, and the sixth skin fiducial 110—are arranged in the second target arrangement on skin of a leg of the second human patient, the second target arrangement defined for a knee surgery and defining locations of the set of skin fiducials 110 relative to anatomical features associated with knee surgery.

### 4.5 Variation: Non-uniform Skin Fiducials

In one implementation, the set of skin fiducials 110 is configured to be individually identifiable in images captured by the sensor suite.

In particular, in this implementation, the spherical section 114 of the skin fiducial 110 can include a visual characteristic that distinguishes the spherical section 114 from spherical sections 114 of other skin fiducials 110 in the set of skin fiducials 110. For example, the spherical section 114 can include a color, a color pattern, a spectral characteristic, or another optical feature detectable in image data captured by one or more optical tracking sensors 122 arranged within the operating space.

In this implementation, the optical tracking sensors 122 can include one or more visible-light imaging sensors configured to capture color image data of a region of the patient's body including the set of skin fiducials 110. The computer system 150 can access the color image data and associate each skin fiducial 110 with an identifier based on the visual characteristic of the corresponding spherical section 114. For example, the computer system 150 can assign a unique identifier to each skin fiducial 110 based on a detected color of the spherical section 114. Therefore, the computer system 150 can track individual skin fiducials 110 across image frames captured by the optical tracking sensors 122 without relying solely on spatial proximity or relative geometric arrangement of the set of skin fiducials 110.

By identifying individual skin fiducials 110, the computer system 150 can improve robustness of fiducial tracking during execution of a medical procedure. For example, the computer system 150 can maintain correspondence between individual skin fiducials 110 detected in image data captured by the optical tracking sensors 122 and corresponding fiducials detected in a medical image captured by an imaging scanner. The computer system 150 can also maintain consistent identification of individual skin fiducials 110 when portions of the patient's body move, when one or more fiducials become temporarily occluded, or when the set of skin fiducials 110 includes a larger number of fiducials arranged across a region of the patient's body. By enabling identification of individual skin fiducials 110, spherical sections 114 of the set of skin fiducials 110 can: reduce ambiguity in correspondence between fiducials detected in different sensing modalities; and improve reliability of spatial registration between medical image data and the patient's anatomy.

In one variation, the spherical sections 114 of the set of skin fiducials 110 include different optical materials configured to produce distinguishable responses under illumination by one or more optical tracking sensors 122. For example, different spherical sections 114 can include coatings configured to reflect different wavelengths of light or to emit distinguishable fluorescence responses when illuminated. The optical tracking sensors 122 can capture image data corresponding to these spectral responses, and the computer system 150 can associate the spectral responses with identifiers corresponding to individual skin fiducials 110. Therefore, the computer system 150 can distinguish among multiple skin fiducials 110 based on spectral characteristics of the spherical sections 114 while maintaining accurate spatial localization of each skin fiducial 110 during operation of the medical projection system 120.

### 5. Projection System

Generally, the projection system 120 includes: an optical sensor 122 (hereinafter “an optical tracking sensor”)—such as including an infrared sensor 124, a depth sensor 126, etc.—configured to capture images of a region of a body of the human patient, the region of the body including the set of skin fiducials 110; a projector 130 configured to project visual overlays of three-dimensional medical images of the human patient onto the region of the body; and a computer system 150. The computer system 150 is configured to: detect the set of skin fiducials 110 in an image captured by the optical tracking sensor 122; derive a set of spatial coordinates of the set of skin fiducials 110 on the region of the body relative the projector 130 based on features extracted from the image; access a three-dimensional medical image of the region of the body captured by an intraoperative scanner arranged in the operating space, the three-dimensional medical image depicting a set of anatomical features of the human patient corresponding to the region of the body; detect a constellation of skin fiducials 110 in the three-dimensional medical image, the constellation of skin fiducials 110 representing the set of skin fiducials 110 on the body; calculate a transform between the three-dimensional medical image and the image by aligning the constellation of skin fiducials 110 in the three-dimensional medical image to the set of spatial coordinates to locate features of the three-dimensional medical image in the operating space; generate a visual overlay based on the set of spatial coordinates and the transform; and trigger the projector 130 to project the visual overlay onto the region of the body during the medical procedure.

In one implementation, the projection system 120 further includes a housing 142 transiently arranged within the operating space. In this implementation, the optical tracking sensor 122 and the projector 130 are rigidly integrated within the housing 142, such that the optical tracking sensor 122 is positionally fixed relative the projector 130. The optical tracking sensor 122 defines a first field of view facing the region of the body and the projector 130 defines a second field of view facing the region of the body and overlapping the first field of view, such that the projection system 120 defines a composite field of view defined by overlapping regions of the first field of view and the second field of view.

Furthermore, in one implementation, the projection system 120 can include a mobile cart 140 transiently arranged within the operating space. In this implementation, the housing 142—including the optical tracking sensor 122 and the projector 130 rigidly integrated within the housing 142—is mounted to the mobile cart 140 and the computer system 150 is arranged or installed within the mobile cart 140.

### 5.1 Infrared Sensor+Depth Sensor

In one implementation, the optical tracking sensor 122 is an infrared sensor 124 configured to capture infrared images of the region of the body.

In this implementation, the projection system 120 can further include a depth sensor 126 configured to capture depth images of the region of the body. The computer system 150 can then: detect the set of skin fiducials 110 in infrared images captured by the infrared sensor 124; and generate a surface model (or “3D mesh”) of the region of the body based on features extracted from depth images captured by the depth sensor 126 (as described further below).

### 5.1.1 Infrared Camera Setup

In one implementation, the optical tracking sensor 122 includes an infrared camera configured to capture infrared images of regions of the patient's body to support optical tracking of skin fiducials 110 arranged on these regions of the patient's body.

In one example, the infrared camera includes a first infrared imaging sensor and a second infrared imaging sensor arranged at known positions relative to one another and oriented toward a common field of view within the operating space. The infrared camera further includes one or more infrared illumination sources configured to emit infrared light toward the region of the patient's body. In this example, the computer system 150 accesses infrared images captured by the first infrared imaging sensor and infrared images captured by the second infrared imaging sensor and identifies bright regions corresponding to spherical sections 114 of the set of skin fiducials 110 depicted within each infrared image. The computer system 150 then: calculates image-space center locations of these spherical sections 114 within the infrared images; accesses a set of calibration data defining spatial relationships between the first infrared imaging sensor and the second infrared imaging sensor; and calculates a set of spatial coordinates of the spherical sections 114 relative the projection system 120 based on geometric relationships between image-space center locations detected in infrared images and the set of calibration data. By leveraging infrared images captured by both the first infrared imaging sensor and the second infrared imaging sensor—which observe spherical sections 114 of the set of skin fiducials 110 from different viewpoints—the computer system 150 can triangulate the set of spherical sections 114 within the operating space and thus enable continuous tracking of the set of skin fiducials 110 during execution of the medical procedure.

### 5.2 Variation: Depth Sensor

In one variation, the projection system 120 includes a depth sensor 126 (e.g., a color and depth sensor 126) configured to capture depth data representing the region (i.e., the region of interest) of the body of the human patient. In this variation, the computer system 150 is configured to both: detect the set of skin fiducials 110 in depth images captured by the depth sensor 126; and generate a surface model (or “3Dmesh”) of the region of the body based on features extracted from these depth images.

In particular, in this variation, the projection system 120 omits a dedicated infrared sensor 124 and includes one or more optical tracking sensors 122 configured to capture image data of the patient's body leveraged for both detecting the set of skin fiducials 110 and surface mapping of the patient's body. For example, the projection system 120 can include a color imaging sensor, a depth sensor 126, an RGB-D sensor, or another optical sensing device configured to capture both visual image data and depth information corresponding to the region of the patient's body. In this variation, the optical tracking sensor 122 captures images depicting surface geometry of the patient's body and the set of skin fiducials 110 affixed to the patient's skin. The computer system 150 accesses these images and detects the set of skin fiducials 110 within these images based on visual characteristics of the set of skin fiducials 110 such as color, shape, intensity contrast, or other optical features associated with spherical sections 114 of the set of skin fiducials 110.

In this implementation, the computer system 150 processes image data captured by the optical tracking sensor 122 to calculate spatial coordinates of the set of skin fiducials 110 relative to the projection system 120 and to generate a surface model of the region of the patient's body. For example, the depth sensor 126 or RGB-D sensor can capture depth images corresponding to surface geometry of the patient's skin while the color imaging sensor captures visual image data depicting the spherical sections 114 of the set of skin fiducials 110. The computer system 150 can associate image features corresponding to the set of skin fiducials 110 with corresponding depth values captured by the optical tracking sensor 122 and calculate spatial positions of the set of skin fiducials 110 relative to the medical projection system 120. The computer system 150 can then generate a projection overlay corresponding to anatomical information derived from a medical image and can control the projector 130 system to project the projection overlay onto the region of the patient's body based on the spatial positions of the set of skin fiducials 110 and the surface model generated from the captured depth information.

By capturing both fiducial image data and surface geometry via the same optical tracking sensor 122, the projection system 120 can reduce hardware complexity while enabling both: detection of skin fiducials 110 on the patient's skin and depicted in images captured by the optical tracking sensor 122; and generation of the surface model representing the region of the patient's body. The projection system 120 can thus maintain alignment between projected anatomical overlays and corresponding anatomical structures of the patient while reducing system cost, simplifying calibration procedures, and reducing integration complexity within the operating space.

### 6. Skin Fiducial Placement

Generally, the computer system 150 can detect an array of skin fiducials 110—transiently affixed to skin of a human body—arranged in a particular arrangement on the human body.

In one implementation, during a setup period, the computer system 150 can access a set of skin fiducial 110 templates defining arrangements of skin fiducials 110 on the human body for different medical procedures. In particular, the computer system 150 can access a particular skin fiducial 110 template, in the set of skin fiducial 110 templates, corresponding to a medical procedure type of a medical procedure scheduled for a patient. The computer system 150 can then provide the particular skin fiducial 110 template to a user (e.g., a technician, a medical professional, a surgeon) in preparation for the scheduled medical procedure. For example, during the setup period, the computer system 150 can: receive confirmation of a first medical procedure type, in a set of medical procedure types (e.g., a knee replacement, an appendectomy, a biopsy, a heart valve repair), for a first medical procedure scheduled during an operation period succeeding the setup period; access a template database including a set of fiducial templates defined for the set of medical procedure types; and retrieve a first fiducial template, in the set of fiducial templates, associated with the first medical procedure type in the template database. The computer system 150 can then: generate a prompt to arrange a first array of skin fiducials 110 on the patient's body according to the first fiducial template; append the prompt with the first fiducial template; and transmit the prompt to a computing device accessed by a medical professional affiliated with the first medical procedure, such as prior to a start time of the first medical procedure.

In particular, in one example, the computer system 150 can: receive confirmation of a knee replacement surgery scheduled for a first patient; retrieve a first fiducial template—such as including a first array of skin fiducials 110 arranged on skin proximal and/or about a human knee in a first arrangement —associated with knee replacement surgery in the template database; receive confirmation of a rotator cuff repair scheduled for a second patient; retrieve a second fiducial template—such as including a second array of skin fiducials 110 arranged on skin proximal and/or about a human shoulder in a second arrangement—associated with rotator cuff repair surgery in the template database; receive confirmation of a pacemaker implantation scheduled for a third patient; retrieve a third fiducial template—such as including a third array of skin fiducials 110 arranged on skin proximal and/or about a human heart in a third arrangement-associated with pacemaker implantation surgery in the template database; etc.

Additionally, in this implementation, the computer system 150 can access the set of fiducial templates—stored in the template database—each defining a particular array of skin fiducials 110 including a set of primary skin fiducials 110 and a set of secondary skin fiducials 110. In particular, the computer system 150 can access a skin fiducial 110 template—defined for a particular medical procedure type in a set of medical procedure types—defining an arrangement of skin fiducials 110 including: a first set of primary skin fiducials 110 arranged proximal (e.g., with a threshold distance of) a primary surgical site defined for the particular medical procedure type; and a set of secondary skin fiducials 110 arranged on the skin relatively further from the primary surgical site and, therefore, less likely to require removal and/or relocation during performing of a medical procedure of the particular medical procedure type (e.g., by a medical professional).

For example, for an appendectomy, the computer system 150 can access a skin fiducial 110 template—defined for an appendectomy surgery —defining an arrangement of a set of skin fiducials 110 including: a set of primary skin fiducials 110 arranged in a first arrangement on the skin proximal and/or about a human appendix; and a set of secondary skin fiducials 110 arranged in a second arrangement on the skin about the first set of primary skin fiducials 110 and further from the human appendix than the first set of primary skin fiducials 110.

By including this secondary set of skin fiducials 110, the computer system 150 can register a patient's medical image to the patient's body in the physical space—via the set of skin fiducials 110—regardless of whether primary skin fiducials 110 require removal during surgery, such as in order to enable access to the primary surgical site. Furthermore, the set of secondary skin fiducials 110 can include at least a target quantity of skin fiducials 110, such that secondary skin fiducials 110 may be removed-intentionally and/or unintentionally-during surgery without reducing robustness of detection and therefore enabling registration of a medical image to the patient's body.

### 7. IR Image: Confirm Skin Fiducial Arrangement

Block S110 of the method S100 recites: accessing an infrared image of a region of a body and captured by an infrared sensor 124 arranged at a first location in an operating space. Furthermore, Block S112 of the method S100 recites: detecting a set of skin fiducials 110 arranged in a first arrangement on the body in the infrared image, each skin fiducial 110, in the set of skin fiducials 110, including a base section 112, an adhesive layer 116 applied to the base section 112 and configured to transiently affix the base section 112 to skin of the body, and a spherical section 114 coupled to the base section 112 and including a retroreflective surface.

Generally, the computer system 150 can access an IR image-captured via an IR camera installed in the operating space—of an array of skin fiducials 110 arranged on the skin of a patient during the setup period and prior to an operating period.

In particular, the computer system 150 can: receive confirmation of setup of an array of skin fiducials 110 on a patient's body in preparation for a medical procedure of a first medical procedure type; access an IR image-captured by one or more IR cameras arranged in the operating space-of a region of a patient's body and depicting the array of skin fiducials 110; detect the array of skin fiducials 110 in the IR image based on detection of reflected light exceeding a threshold intensity at each skin fiducial 110, in the array of skin fiducials 110, in the IR image; and interpret a 3D location (e.g., 3D coordinates) of each skin fiducial 110, in the array of skin fiducials 110, in the IR image.

In one implementation, the computer system 150 can then leverage detection of the set of skin fiducials 110 in the IR image to confirm an operative setup prior to the operating period. In particular, in one example, the computer system 150 can: retrieve a fiducial template—corresponding to the first medical procedure type and defining a target arrangement of skin fiducials 110—from the template database; interpret a first arrangement of the set of skin fiducials 110 on the patient's body based on 3D locations of each skin fiducial 110, in the array of skin fiducials 110, derived from the IR image; and characterize a difference between the first arrangement of the set of skin fiducials 110 on the patient's body and the target arrangement of skin fiducials 110 defined by the fiducial template for the first medical procedure type. Then, in response to the difference exceeding a threshold difference, the computer system 150 can: flag the first arrangement of the set of skin fiducials 110 on the patient's body as a discrepancy between the first arrangement and the target arrangement; generate a notification indicating the detected discrepancy of the set of skin fiducials 110; and transmit the notification to a computing device accessed by a medical professional affiliated with the first medical procedure type for the patient.

Additionally or alternatively, in another example, the computer system 150 can: interpret a first arrangement of the set of skin fiducials 110 on the patient's body based on 3D locations of each skin fiducial 110, in the array of skin fiducials 110, derived from the IR image; and, based on the first arrangement, derive a set of distances corresponding to distances between each skin fiducial 110 in the set of skin fiducials 110 in the first arrangement on the patient's body. Then, in response to a first distance in the set of distances—between a first skin fiducial 110 and a second skin fiducial 110 arranged on the patient's body—falling below a threshold distance, the computer system 150 can: flag the first skin fiducial 110 and the second skin fiducial 110 as a setup error; generate a prompt—indicating the setup error—to relocate the first and/or second skin fiducial 110 to increase the first distance between the first and second skin fiducials 110 accordingly; and transmit the prompt to a computing device accessed by a medical professional affiliated with the first medical procedure type for the patient.

Additionally or alternatively, in yet another example, the computer system 150 can flag a setup error based on placement of skin fiducials 110 on regions of skin corresponding to “fleshy” or “compliant” skin that may be more susceptible to movement during surgery. Therefore, the computer system 150 can promote placement of skin fiducials 110 on regions of human skin that is more rigid—and therefore less likely to exhibit movement during execution of a medical procedure on the patient—thereby improving alignment of a projected medical image onto the patient's body based on known locations of skin fiducials 110 on the patient's skin.

Additionally or alternatively, in yet another example, the computer system 150 can flag a setup error based on placement of skin fiducials 110 within a particular region of the body that may interfere with a medical procedure of a particular medical procedure type defined for the patient. For example, in response to detecting a skin fiducial 110 in a location intersecting a cutting pathway—corresponding to a surgical incision—defined for a particular medical procedure type, the computer system 150 can flag a setup error associated with interference of the skin fiducial 110 with a medical procedure of the medical procedure type.

Additionally or alternatively, in yet another example, the computer system 150 can flag a setup error based on placement of the set of skin fiducials 110 in a symmetrical arrangement on the patient's skin, which may lead to errors in detection of an orientation of the set of skin fiducials 110 on the patient's skin. In particular, in this example, the computer system 150 can: detect the set of skin fiducials 110 in a symmetrical arrangement—such as arranged in a line, in a triangular pattern, in a rectangular pattern, etc.—on the patient's skin; and, in response to detection of the set of skin fiducials 110 in the symmetrical arrangement, flag a setup error associated with an undetectable orientation of the set of skin fiducials 110 on the patient's skin.

In this implementation, in response to verifying a setup of the set of skin fiducials 110 in a particular arrangement on the patient's body—such as characterized by absence of detected errors in the setup—the computer system 150 can: generate a notification indicating verification of the setup for the set of skin fiducials 110 for the particular medical procedure type; and transmit the notification to the medical professional affiliated with the medical procedure of the particular medical procedure type for this patient.

### 7.1 Skin Fiducial Detection: Spherical Center Estimation

In one implementation, the computer system 150 can access the infrared images captured by the infrared sensor 124 and identify bright regions corresponding to the set of spherical sections 114 of the set of skin fiducials 110. The computer system 150 can then analyze pixel intensities within each bright region and calculate an image-space center location of the corresponding spherical section 114 based on a distribution of intensity values across the bright region.

For example, the computer system 150 can: fit a circular model, an elliptical model, a centroid model, and/or another geometric or intensity-based model to the bright region; and calculate a center location of the spherical section 114 at a location between adjacent pixel centers in the infrared image. Because the spherical section 114 spans multiple pixels in the infrared image and because the retroreflective material produces a high-contrast image feature, the computer system 150 can calculate the center location of the spherical section 114 with sub-pixel precision relative to the pixel grid of the infrared image.

### 8. Interoperative Imaging+Skin Fiducial Detection

Block S120 of the method S100 recites: accessing a three-dimensional medical image of the region of the body, the three-dimensional medical image captured by an intraoperative scanner arranged at a second location in the operating space. Furthermore, Block S122 recites detecting a constellation of skin fiducials 110 in the three-dimensional medical image, the constellation of skin fiducials 110 representing the set of skin fiducials 110 on the body.

Generally, the computer system 150: accesses a 3D medical image of a region of the patient's body captured via an intraoperative scanner in the operating space; and detects the array of skin fiducials 110—applied to skin of the patient's body—in the 3D medical image. The system can then leverage detection of these skin fiducials 110 in the 3D medical image—in combination with detection of skin fiducials 110 in the IR image—to locate data extracted from the 3D medical image onto the patient's body in the operating space.

In particular, as described above, the computer system 150 can: access an IR image of a region of a patient's body and depicting the array of skin fiducials 110; and interpret a set of location coordinates for each skin fiducial 110, in the array of skin fiducials 110, on the patient and in the operating space in the IR image. For example, the set of skin fiducials 110 can include: a first skin fiducial 110 transiently affixed to a first location within the region of the body; a second skin fiducial 110 transiently affixed to a second location within the region of the body; and a third skin fiducial 110 transiently affixed to a third location within the region of the body. In this example, the computer system 150 can: access an infrared image of the region of the patient's body; detect the first skin fiducial 110 at the first location; detect the second skin fiducial 110 at the second location; detect the third skin fiducial 110 at the third location; calculate a first set of location coordinates corresponding to the first location of the first skin fiducial 110 based on a first set of features extracted from the infrared image; calculate a second set of location coordinates corresponding to the second location of the second skin fiducial 110 based on a second set of features extracted from the infrared image; and calculate a third set of location coordinates corresponding to the third location of the third skin fiducial 110 based on a third set of features extracted from the infrared image.

Then, the computer system 150 can: access an X-ray or CT scan captured by an intraoperative scanner located in the operating space while the array of skin fiducials 110 are applied to the patient's body; detect a constellation of skin fiducials 110—corresponding to the array of skin fiducials 110 affixed to the patient's body—in the X-ray or CT scan; and, based on correspondence between the constellation of skin fiducials 110 in the 3D medical image and the array of skin fiducials 110 in the IR image, virtually align the 3D image to the patient's body.

In one implementation, the computer system 150 can verify whether at least a threshold quantity of skin fiducials 110, in the set of skin fiducials 110 applied to the patient's body, are detectable in the 3D medical image of the patient's body. In this implementation, in response to detecting less than a threshold quantity of skin fiducials 110 in the 3D medical image, the computer system 150 can: flag the 3D medical image; generate a notification indicating detection of less than a threshold quantity of skin fiducials 110 in the 3D medical image; append the notification with a prompt to re-capture the 3D medical image; and transmit the notification to an operator associated with the medical procedure and/or capturing of the 3D medical image. Therefore, the computer system 150 can promote capture of 3D medical images depicting a sufficient quantity of skin fiducials 110 to enable mapping of the 3D medical image—in combination with the IR image—to the patient's body.

### 8.1 Registration: IR Image+3D Medical Image

Block S130 of the method S100 recites: calculating a transform between the three-dimensional medical image and the infrared image by aligning the set of skin fiducials 110 in the three-dimensional medical image to the set of location coordinates of the set of skin fiducials 110 in the operating space to locate features of the three-dimensional medical image in the operating space.

Generally, the computer system 150 can leverage detection of skin fiducials 110 in both the IR image and the 3D medical image to map 3D image data —extracted from the 3D medical image and representing a region of the patient's body depicted in the 3D medical image—onto the patient's physical body.

In particular, the computer system 150 can: access an IR image—captured by an IR camera arranged in the operating space—of a region of a patient's body and depicting an array of skin fiducials 110; detect the array of skin fiducials 110 in the IR image based on detection of reflected light exceeding a threshold intensity at each skin fiducial 110 in the IR image; interpret a set of location coordinates for each skin fiducial 110, in the array of skin fiducials 110, on the patient and in the operating space in the IR image; access a 3D medical image (e.g., an X-ray, a CT scan)—captured by an intraoperative scanner in the operating space—of the region of the of the patient's body and depicting the array of skin fiducials 110; detect a constellation of skin fiducials 110—corresponding to the array of skin fiducials 110 on the patient's skin—in the 3D medical image; and calculate a transformation matrix that aligns the constellation of skin fiducials 110 in the 3D medical image to the patient's body in the operating space based on the set of location coordinates for each skin fiducial 110 in the array of skin fiducials 110 on the patient's body.

The computer system 150 can thus align the constellation of skin fiducials 110 in the 3D image to the array of skin fiducials 110 depicted in the IR image and arranged on the patient's body in the operating space.

### 8.2 Patient Projection

Block S140 of the method S100 recites: based on the location of the projector 130, the set of location coordinates, and the transform, generating a visual overlay of the three-dimensional medical image configured for projection onto the region of the human patient's body, the constellation of skin fiducials 110 depicted in the three-dimensional medical image aligned to the set of skin fiducials 110 applied to the human patient's body. Furthermore, Block S142 of the method S100 recites: triggering the projector 130 to project the visual overlay onto the region of the body in the operating space.

Generally, the computer system 150 can: generate a projection overlay of the 3D medical image—including a constellation of skin fiducials 110 representing a set of skin fiducials 110 arranged on the patient's body—captured for the patient; and trigger a projector 130—arranged within the operating space at a known location and defining a field of view facing the patient's body—to project the projection overlay onto the patient's body, such that the constellation of skin fiducials 110 in the 3D medical image align with the set of skin fiducials 110 arranged on the patient's body.

In particular, the computer system 150 can leverage detection of the set of skin fiducials 110 on the patient's body—detected in both the IR image (and/or RGB image) and the 3D medical image (e.g., CT scan, X-ray)—to generate and project a projection overlay of the 3D medical image onto the patient's body such that features depicted in the 3D medical image align with these features of the patient's body.

In one example, the computer system 150 can: access a first location of an IR camera (or IR sensor) in the operating space; access a second location of a 3D camera (e.g., a depth sensor 126) in the operating space; access a third location of a projector 130 in the operating space; and generate a virtual representation of the operating space based on the first location of the IR camera, the second location of the 3D camera, and the third location of the projector 130. The computer system 150 can then: access a 3D image (e.g., a depth image) of a region of the patient's body (e.g., a torso, a chest, a head, a trunk) captured by the 3D camera; define a 3D mesh representing a surface of the region of the patient's body based on the 3D image; represent the 3D mesh in the virtual representation of the operating space, such as positioned correctly relative the projector 130; detect a set of skin fiducials 110—arranged on the region of the patient's body—in the 3D image and/or in an IR image captured by the IR camera; for each skin fiducial 110, in the set of skin fiducials 110, identify a set of location coordinates representing a location of the skin fiducial 110 on the region of the patient's body; and represent the set of skin fiducials 110 on the 3D mesh based on the set of location coordinates of each skin fiducial 110 in the set of skin fiducials 110.

Then, the computer system 150 can: access a 3D medical image (e.g., an X-ray, a CT scan)—captured by an intraoperative scanner in the operating space —of the region of the patient's body and depicting the set of skin fiducials 110; and locate the 3D medical image relative the mesh in the virtual representation of the operating space by overlaying the set of skin fiducials 110 in the 3D medical image with the set of skin fiducials 110 represented in the 3D mesh (and detected in the IR image). Finally, the computer system 150 can: access a field of view of the projector 130; calculate a transform of the 3D medical image onto the (non-planar) surface of skin of the region of the patient's body from the 3D mesh; based on the field of view and the transform, generate a projection overlay—of the 3D medical image—for projecting onto the patient's body, such that the 3D medical image aligns to the region of the patient's body via the set of skin fiducials 110; and trigger the projector 130 to project the projection overlay onto the patient's body accordingly. In particular, the computer system 150 can generate the visual overlay of the 3D medical image—configured to conform to curvature of the surface of the region of the body during projection onto the region of the body —based on known locations of the projector 130 and sensors, the set of location coordinates of the set of skin fiducials 110 derived from the infrared image (or other image type), the transform, and the 3D mesh.

For example, the computer system 150 can trigger the projector 130 to: project a projection overlay of a CT scan—depicting the patient's chest in preparation for heart surgery—onto the patient's chest, such that the projection overlay on the patient's body includes a surgical boundary about the patient's heart, a representation of the patient's heart and/or valves connected to the heart, etc.; project a projection overlay of a CT scan—depicting the patient's head in preparation for brain surgery—onto the patient's head, such that the projection overlay on the patient's head includes a boundary of a tumor in the patient's brain, representations of different regions of the patient's brain to avoid and/or cut into during surgery, etc.; and project a projection overlay of a CT scan—depicting the patient's torso in preparation for spinal surgery—onto the patient's torso, such that the projection overlay on the patient's head includes a representation of the patient's spine, a region of interest on the patient's spine, adjacent organs and/or tissues to avoid during surgery, etc.

### 8.2.1 Projection Calculation

In one implementation, the computer system 150 registers a three-dimensional medical image of a region of a patient's body to the physical anatomy of the patient based on detection of the set of skin fiducials 110 affixed to the patient's skin. The intraoperative scanner (e.g., a CT scanner) can capture the three-dimensional medical image depicting the region of the patient's body and the set of skin fiducials 110 arranged on the patient. The optical tracking sensor (e.g., infrared sensor 124, depth sensor 126) arranged in the operating space can capture images of the region of the patient's body depicting the spherical sections of the set of skin fiducials 110. The computer system 150 can then detect the set of skin fiducials 110 in these images and calculate a set of spatial coordinates of spherical sections of the set of skin fiducials 110 relative a sensor coordinate frame associated with the optical tracking sensor 122. The computer system 150 can also detect a constellation of skin fiducials in the three-dimensional medical image and calculate corresponding coordinates of the set of skin fiducials 110 in a scanner coordinate frame associated with the intraoperative scanner.

The computer system 150 can calculate a first transform between the scanner coordinate frame of the three-dimensional medical image and the sensor coordinate frame associated with the optical tracking sensor 122. In particular, the computer system 150 can align the constellation of skin fiducials detected in the three-dimensional medical image with the set of spatial coordinates of corresponding skin fiducials detected in images captured by the optical tracking sensor 122. Based on this alignment, the computer system 150 can calculate the first transform that maps anatomical features represented in the three-dimensional medical image to corresponding locations on the patient's body—expressed in the sensor coordinate frame—in the operating space.

The computer system 150 can then calculate a second transform between the sensor coordinate frame and a projector coordinate frame associated with the projector 130. In particular, the computer system 150 can access a set of calibration parameters defining a spatial pose of the projector 130 relative the optical tracking sensor 122. Based on the set of calibration parameters, the computer system 150 can calculate the second transform configured to convert spatial coordinates represented in the sensor coordinate frame to corresponding coordinates represented in the projector coordinate frame. The computer system 150 can then leverage the second transform to generate a projection overlay configured for projection by the projector 130.

In particular, the computer system 150 can generate a projection overlay by rendering the projection overlay in the projector coordinate frame based on the first transform and the second transform. The computer system 150 can convert coordinates of anatomical features represented in the three-dimensional medical image to corresponding spatial coordinates associated with the patient's body expressed in the sensor coordinate frame based on the first transform. The computer system 150 can then convert these spatial coordinates to projector coordinates based on the second transform. The projector 130 can then project the projection overlay onto the region of the patient's body, such that anatomical features depicted in the projection overlay align with corresponding anatomical features of the patient's body during execution of the medical procedure.

Furthermore, the depth sensor 126 arranged in the operating space can capture depth images representing a surface of the region of the patient's body on which the set of skin fiducials 110 are arranged. The computer system 150 can then: access these depth images captured by the depth sensor; generate a three-dimensional surface model of the region of the patient's body based on depth values represented in the depth images; and represent the surface model in the sensor coordinate frame based on spatial coordinates of the set of skin fiducials 110 detected in images captured by the optical tracking sensor 122 and a set of calibration parameters defining spatial relationships between the depth sensor 126 and the optical tracking sensor 122 (e.g., in the implementation in which the optical tracking sensor 122 is distinct from the depth sensor 126). The computer system 150 can then project anatomical features represented in the three-dimensional medical image onto the surface model based on the first transform between the scanner coordinate frame and the sensor coordinate frame. In particular, the computer system 150 can convert coordinates of anatomical features represented in the three-dimensional medical image to corresponding coordinates associated with the surface model of the patient's body. The computer system 150 can therefore identify locations on the surface of the patient's body corresponding to anatomical features represented in the three-dimensional medical image.

The computer system 150 can then convert coordinates of these locations of anatomical features on the surface model to projector coordinates based on the second transform between the sensor coordinate frame and the projector coordinate frame. Based on these projector coordinates, the computer system 150 can generate the projection overlay configured for projection via the projector 130 onto the region of the patient's body. The projector 130 can then project the projection overlay onto the region of the patient's body, such that anatomical features represented in the projection overlay align with corresponding anatomical features of the patient's body despite curvature of the patient's body surface.

### 8.2.2 Variation: Perspective Transformation for Surgeon Viewpoint

In one variation, the computer system 150 can generate the projection overlay based on a viewing perspective of a surgeon performing the medical procedure. The optical tracking sensor 122—including the infrared sensor 124 and/or the depth sensor 126—arranged in the operating space can capture images of a region of the patient's body including the set of skin fiducials 110. The computer system 150 can detect the set of skin fiducials 110 in these images and calculate spatial coordinates of the spherical sections of the set of skin fiducials 110 relative a sensor coordinate frame associated with the optical tracking sensor 122. The computer system 150 can also detect the constellation of skin fiducials in the three-dimensional medical image and calculate corresponding coordinates of the set of skin fiducials 110 in a scanner coordinate frame associated with the intraoperative scanner. The computer system 150 can then calculate a first transform between the scanner coordinate frame and the sensor coordinate frame based on alignment of the constellation of skin fiducials detected in the three-dimensional medical image with corresponding spatial coordinates of the set of skin fiducials 110 detected in images captured by the optical tracking sensor 122.

In this variation, one or more additional optical sensors arranged in the operating space can capture additional images depicting the surgeon located near the patient. The computer system 150 can detect reference features associated with the surgeon in these images, such as a location of the surgeon's head, eyes, or a head-mounted device worn by the surgeon. Based on these detected features, the computer system 150 can calculate a spatial location of the surgeon relative the region of the patient's body and generate a second transform configured to convert coordinates of the region of the patient's body from the sensor coordinate frame to a surgeon coordinate frame corresponding to a viewpoint of the surgeon. The computer system 150 can therefore represent coordinates of the region of the patient's body and the set of skin fiducials 110 from the viewpoint of the surgeon.

The computer system 150 can then generate a third transform configured to convert coordinates represented in the surgeon coordinate frame to a projector coordinate frame associated with the projector 130. Based on the first transform, the second transform, and the third transform, the computer system 150 can generate the projection overlay configured for projection via the projector 130 onto the region of the patient's body. In particular, the computer system 150 can convert coordinates of anatomical features represented in the three-dimensional medical image to corresponding spatial coordinates expressed in the sensor coordinate frame based on the first transform, convert these spatial coordinates to the surgeon coordinate frame based on the second transform, and convert these coordinates to projector coordinates based on the third transform. The projector 130 can then project the projection overlay onto the region of the patient's body such that anatomical features represented in the projection overlay appear aligned with corresponding anatomical features of the patient's body when viewed from the surgeon's position during execution of the medical procedure.

### 9. Results

Generally, once the calculated projection overlay of the 3D medical image is projected onto the patient's body in the operating space, the computer system 150 can detect an error in this projection and selectively notify the medical professional accordingly.

In particular, the computer system 150 can: include the set of skin fiducials 110 in the calculated projection overlay that is projected onto the patient's body; detect these projected skin fiducials 110 on the patient's body in images (e.g., RGB images) captured by an optical tracking sensor 122, such as integrated in an RGB camera installed in the operating space; detect the (physical) set of skin fiducials 110 arranged on the patient's body in these images; and, for each skin fiducial 110, in the set of skin fiducials 110, calculate an offset between the actual skin fiducial 110 on the patient's body and the projected skin fiducial 110 projected onto the patient's body.

Then, in response to the offset exceeding a threshold offset, the computer system 150 can: flag the calculated projection for misalignment; generate a notification indicating misalignment of the projection overlay onto the patient's body; append the notification with a prompt to recalibrate the projection overlay via execution of the methods and techniques described above; and transmit the prompt to a computing device accessed by the medical professional. Additionally or alternatively, the computer system 150 can trigger the projector 130 to project an error message—indicating misalignment of the projection overlay onto the patient's body—onto the patient's body for viewing by the medical professional. Alternatively, in response to each offset corresponding to a rigid transformation of the projection overlay relative the patient's body—such that the operating table moved within the operating space (e.g., relative the projector 130) while the patient's body exhibits no deformation—the computer system 150 can recalculate the projection overlay accordingly to shift the projection overlay onto the patient's body on the relocated operating table.

Alternatively, in response to the offset falling below the threshold offset, the computer system 150 can: verify the projection overlay as accurate; and generate and transmit a notification-indicating the projection overlay as accurate —to the medical professional. Similarly, the computer system 150 can also trigger the projector 130 to project a verification message—indicating verification of the projection overlay onto the patient's body—onto the patient's body for viewing by the medical professional.

### 10. Real-Time Updates to the Projection Overlay

During execution of the medical procedure, the computer system 150 can continuously monitor a position of the patient's body in the operating space and update the projected visual overlay accordingly. In particular, the computer system 150 can continue accessing additional infrared images and/or depth images captured by one or more optical tracking sensors 122—such as including the infrared sensor 124 and/or the depth sensor 126 of the projection system 120—arranged in the operating space.

The computer system 150 can detect the set of skin fiducials 110 in each newly captured infrared image and extract features corresponding to the spherical sections of the skin fiducials. Based on these features, the computer system 150 can recalculate spatial coordinates of the set of skin fiducials 110 in the operating space and identify any displacement of skin fiducials 110 relative the projection system 120. For example, the computer system 150 can detect movement of the patient's body resulting from respiration, minor repositioning of the patient during the procedure, deformation of the patient's skin, or interaction between a medical professional and the patient's body.

In response to detecting movement of the set of skin fiducials 110, the computer system 150 can update the transform between the three-dimensional medical image and the region of the patient's body in the operating space. The computer system 150 can then regenerate the projection overlay based on the updated spatial coordinates and the updated transform. The computer system 150 can subsequently trigger the projector 130 to project the updated visual overlay onto the region of the patient's body such that anatomical features depicted in the three-dimensional medical image remain aligned with corresponding anatomical features of the patient's body despite movement of the patient.

In one implementation, the computer system 150 can further access depth data captured by the depth sensor 126 representing a surface geometry of the region of the patient's body. The computer system 150 can update a surface model of the region of the patient's body based on the depth data and generate the projection overlay based on the updated surface model. Accordingly, the computer system can ensure that the projection overlay remains registered to the curved surface of the patient's body while compensating for motion and deformation of the patient's skin during the medical procedure.

Therefore, the projection system 120 can dynamically maintain alignment between projected medical image information and the patient's anatomy throughout the medical procedure without requiring the medical professional to manually re-register the projection system 120.

### 10.1 Variation: Predicted Accuracy

In one variation, the computer system 150 can predict accuracy of the projection overlay and selectively notify the medical professional of changes in accuracy in (near) real-time during execution of a medical procedure on the patient.

In one implementation, the computer system 150 can project an accuracy gauge-depicting a current predicted accuracy of projection—onto the patient's body during a medical procedure. For example, the computer system 150 can predict a varying projection accuracy as a patient breathes—due to expansion and contraction of the patient's lungs—causing movements in the patient's chest and/or other regions of the body. In this example, the computer system 150 can: predict a maximum accuracy when the patient's lungs are contracted and in-between breath cycles; predict a minimum accuracy when the patient's lungs are expanded after breathing air inward; interpret that the patients'lungs are fully contracted at a first time based on proximity of skin fiducials 110, in the set of skin fiducials 110 arranged on the patient's body, such that the set of skin fiducials 110 exhibits a minimum distance between skin fiducials 110 in the set of skin fiducials 110 at the first time; interpret that the patients'lungs are fully expanded at a second time based on proximity of skin fiducials 110, in the set of skin fiducials 110 arranged on the patient's body, such that the set of skin fiducials 110 exhibits a maximum distance between skin fiducials 110 in the set of skin fiducials 110 at the second time; assign the maximum accuracy to the first time and project this maximum accuracy on the patient's body at the first time accordingly; and assign the minimum accuracy to the second time and project this minimum accuracy on the patient's body at the second time accordingly. In this example, the surgeon may therefore elect to perform specific steps of the medical procedure at times corresponding to the maximum accuracy.

### 11. Variation: Instrument Tracking

In one variation, the computer system 150 can track a set of instruments (i.e., surgical instruments) present in the operating space and/or implemented during execution of the medical procedure. Generally, the computer system 150 can maintain and access an instrument library of a set of instruments (e.g., an awl, a tap, a screwdriver, a drill, a dilator, a drill guide) implemented during medical procedures of a set of medical procedure types. In particular, the computer system 150 can: identify a first instrument in the set of instruments; access a first set of instrument characteristics—such as including an instrument geometry, an instrument length, a geometry of a known tracking array defined for the instrument, etc.—defined for the first instrument; and store the first set of instrument characteristics—linked to the first instrument—in the instrument library. The computer system 150 can repeat this process for each other instrument in the set of instruments to maintain the instrument library.

Then, during and/or prior to execution of a medical procedure for a patient, the computer system 150 can identify and track these instruments in the operating space in (near) real-time based on characteristics of the instrument stored in the instrument library. For example, the computer system 150 can: access an image (e.g., an IR image, an RGB image) of the operating space; detect an object in the image based on features extracted from the image; based on characteristics of the object corresponding to a first set of characteristics stored in the instrument library, identify the object a first instrument. The computer system 150 can then leverage the first set of characteristics and features extracted from the image to: detect an end effector of the first instrument in the first image; identify a location —such as characterized by a set of location coordinates—in the operating space and/or relative the overlay projection projected on the patient's body; calculate an axis of the first instrument in the operating space; and, based on the location and axis of the first instrument in the operating space, selectively project additional information-on the projection overlay—associated with manipulation of the first instrument during execution of the medical procedure. For example, the computer system 150 can: project the axis onto the patient's body to guide the medical professional maneuvering the first instrument along the axis; project textual information—such as related to a set of bodily features (e.g., a series of vertebrae, a particular organ, a bone) intersecting a pathway of the instrument; etc.

Additionally or alternatively, the computer system 150 can leverage an orientation of the first instrument—relative the patient's body—in the operating space to render a particular “slice” or subsection of the patient's body represented in the 3D medical image. For example, in response to detecting the first instrument at a first angular position relative the patient's body in the operating space, the computer system 150 can: access the 3D medical image for the patient; extract a first “slice” of the 3D medical image—depicting a “slice” of the patient's body approximately parallel the first instrument (or the axis of the first instrument) in the operating space; and implement the methods and techniques described above to project the first “slice” of the 3D medical image onto the patient's body, such that the medical professional may view this particular “slice” of the patient's body predicted to intersect a pathway of the first instrument.

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

I claim:

1. A surgical tracking system comprising a first skin fiducial, in a set of skin fiducials, configured to transiently affix to skin of a human patient occupying an operating space during a medical procedure, the first skin fiducial comprising:
a base section defining:
an upper surface configured to be non-reflective in an infrared spectrum to reduce interference in optical images depicting the first skin fiducial;
a bottom surface opposite the upper surface and configured to seat flush against skin of the human patient; and
a boss extending upward from the upper surface;
a spherical section:
formed of a retroreflective material configured to reflect incident light for detection of the spherical section in optical images depicting the first skin fiducial;
mechanically coupled to the base section;
defining:
an upper section defining a spherical geometry;
a truncated base defining a base surface configured to mate with the upper surface of the base section; and
a receptacle extending inward from the truncated base and configured to receive and retain the boss of the base section to mechanically couple the spherical section to the base section; and
an adhesive layer:
applied to the bottom surface of the base section;
formed of a pressure-sensitive adhesive material; and
configured to transiently affix the base section to skin of the human patient during the medical procedure.

2. The surgical tracking system of claim 1, further comprising:
a second skin fiducial, in the set of skin fiducials, configured to transiently affix to skin of the human patient during the medical procedure, the second skin fiducial comprising:
a second base section defining:
an upper surface configured to be non-reflective in an infrared spectrum to reduce interference with IR optical tracking;
a bottom surface opposite the upper surface and configured to seat flush against skin of the human patient; and
a boss extending upward from the upper surface;
a second spherical section:
formed of a retroreflective material configured to reflect infrared light;
mechanically coupled to the second base section; and
defining:
an upper section defining the spherical geometry;
a truncated base defining a base surface configured to mate with the upper surface of the second base section; and
a receptacle extending inward from the truncated base and configured to receive and retain the boss of the second base section to mechanically couple the second spherical section to the second base section; and
a second adhesive layer:
applied to the bottom surface of the second base section;
formed of the pressure-sensitive adhesive material; and
configured to transiently affix the second base section to skin of the human patient during the medical procedure;
a third skin fiducial, in the set of skin fiducials, configured to transiently affix to skin of the human patient during the medical procedure, the third skin fiducial comprising:
a third base section defining:
an upper surface configured to be non-reflective in an infrared spectrum to reduce interference with IR optical tracking;
a bottom surface opposite the upper surface and configured to seat flush against skin of the human patient; and
a boss extending upward from the upper surface;
a third spherical section:
formed of a retroreflective material configured to reflect infrared light;
mechanically coupled to the third base section;
defining:
an upper section defining the spherical geometry;
a truncated base defining a base surface configured to mate with the upper surface of the second base section; and
a receptacle extending inward from the truncated base and configured to receive and retain the boss of the third base section to mechanically couple the second spherical section to the second base section; and
a third adhesive layer:
applied to the bottom surface of the third base section;
formed of the pressure-sensitive adhesive material; and
configured to transiently affix the third base section to skin of the human patient during the medical procedure; and
wherein the first skin fiducial, the second skin fiducial, and the third skin fiducial are configured to transiently affix to skin of the human patient in a target arrangement defined for the medical procedure.

3. The surgical tracking system of claim 1:
wherein the base section is formed of a polycarbonate material;
wherein the spherical section is formed of a polymethyl methacrylate material comprising a retroreflective coating comprising glass microbeads; and
wherein the adhesive layer is formed of a pressure-sensitive acrylic material.

4. The surgical tracking system of claim 1:
wherein the set of skin fiducials is configured to transiently affix to skin of the human patient in a target arrangement on a region of skin of the human patient; and
further comprising a projection system comprising:
an optical sensor configured to capture images of a region of a body of the human patient, the region of the body comprising the set of skin fiducials;
a projector configured to project visual overlays of three-dimensional medical images of the human patient onto the region of the body; and
a computer system configured to:
detect the set of skin fiducials in an image captured by the optical sensor;
derive a set of spatial coordinates of the set of skin fiducials on the region of the body relative the projector based on features extracted from the image;
access a three-dimensional medical image of the region of the body captured by an intraoperative scanner arranged in the operating space, the three-dimensional medical image depicting a set of anatomical features of the human patient corresponding to the region of the body;
detect a constellation of skin fiducials in the three-dimensional medical image, the constellation of skin fiducials representing the set of skin fiducials on the body;
calculate a transform between the three-dimensional medical image and the image by aligning the constellation of skin fiducials in the three-dimensional medical image to the set of spatial coordinates to locate features of the three-dimensional medical image in the operating space;
generate a visual overlay based on the set of spatial coordinates and the transform; and
trigger the projector to project the visual overlay onto the region of the body during the medical procedure.

5. The surgical tracking system of claim 4:
wherein the optical sensor comprises an infrared sensor configured to capture infrared images of the region of the body;
wherein the projection system further comprises a depth sensor configured to capture depth images of the region of the body; and
wherein the computer system is configured to:
detect the set of skin fiducials in an infrared image captured by the infrared sensor;
generate a surface model of the region of the body based on depth data captured by the depth sensor; and
generate the visual overlay based on the set of spatial coordinates, the transform, and the surface model, the visual overlay configured to conform to curvature of the surface of the region of the body during projection onto the region of the body.

6. The surgical tracking system of claim 4:
wherein the optical sensor comprises a color and depth camera configured to capture depth data and RGB images representing the region of the body; and
wherein the computer system is configured to:
generate a surface model of the region of the body based on depth data captured by the depth sensor; and
generate the visual overlay based on the set of spatial coordinates, the transform, and the surface model, the visual overlay configured to conform to curvature of the surface of the region of the body during projection onto the region of the body.

7. The surgical tracking system of claim 4:
wherein the projection system comprises a housing transiently arranged within the operating space;
wherein the optical sensor and the projector are rigidly integrated within the housing;
wherein the optical sensor defines a first field of view facing the region of the body;
wherein the projector defines a second field of view facing the region of the body, the second field of view overlapping the first field of view; and
wherein the projection system defines a composite field of view defined by overlapping regions of the first field of view and the second field of view.

8. The surgical tracking system of claim 7:
wherein the projection system further comprises a mobile cart transiently arranged within the operating space;
wherein the housing is mounted to the mobile cart; and
wherein the computer system is located within the mobile cart.

9. The surgical tracking system of claim 1:
wherein the set of skin fiducials comprises the first skin fiducial, a second skin fiducial, and a third skin fiducial transiently affixed to the skin of the human patient in a first target arrangement defined for the medical procedure associated with a first set of anatomical structures; and
further comprising a second set of skin fiducials comprising a fourth skin fiducial, a fifth skin fiducial, and a sixth skin fiducial transiently affixed to the skin of a second human patient in a second target arrangement defined for a second medical procedure associated with a second set of anatomical structures.

10. The surgical tracking system of claim 9:
wherein the first skin fiducial, the second skin fiducial, and the third skin fiducial are arranged in the first target arrangement on skin of a back of the human patient, the first target arrangement defined for a spinal surgery and defining locations of the set of skin fiducials relative vertebrae of a spine of the human patient; and
wherein the fourth skin fiducial, the fifth skin fiducial, and the sixth skin fiducial are arranged in the second target arrangement on skin of a leg of the human patient, the second target arrangement defined for a knee surgery and defining locations of the set of skin fiducials relative anatomical features associated with knee surgery.

11. The surgical tracking system of claim 1, wherein the spherical section is formed of a radiopaque and retroreflective material configured for detection in three-dimensional medical images captured by an intraoperative scanner arranged within the operating space.

12. A surgical tracking system comprising:
a set of skin fiducials configured to transiently affix to skin of a human patient occupying an operating space during a medical procedure, each skin fiducial comprising a retroreflective spherical section configured to reflect incident light; and
a projection system comprising:
an optical sensor configured to capture images of a region of a body of the human patient, the region of the body comprising the set of skin fiducials;
a projector configured to project visual overlays of three-dimensional medical images of the human patient onto the region of the body; and
a computer system configured to:
detect the set of skin fiducials in an image captured by the optical sensor;
derive a set of spatial coordinates of the set of skin fiducials on the region of the body relative the projector based on features extracted from the image;
access a three-dimensional medical image of the region of the body captured by an intraoperative scanner arranged in the operating space, the three-dimensional medical image depicting a set of anatomical features of the human patient corresponding to the region of the body;
detect a constellation of skin fiducials in the three-dimensional medical image, the constellation of skin fiducials representing the set of skin fiducials on the body;
calculate a transform between the three-dimensional medical image and the image by aligning the constellation of skin fiducials in the three-dimensional medical image to the set of spatial coordinates to locate features of the three-dimensional medical image in the operating space;
generate a visual overlay based on the set of spatial coordinates and the transform; and
trigger the projector to project the visual overlay onto the region of the body during the medical procedure.

13. The surgical tracking system of claim 12, wherein each skin fiducial, in the set of skin fiducials, comprises:
a base section defining:
an upper surface configured to be non-reflective to reduce interference in images captured by the optical sensor;
a bottom surface opposite the upper surface and configured to seat flush against skin of the human patient; and
a boss extending upward from the upper surface;
a spherical section:
formed of a retroreflective material configured to reflect incident light output by the optical sensor;
mechanically coupled to the base section; and
defining:
an upper section defining a spherical geometry;
a truncated base defining a base surface configured to mate with the upper surface of the base section; and
a receptacle extending inward from the truncated base and configured to receive and retain the boss of the base section to mechanically couple the spherical section to the base section; and
an adhesive layer:
applied to the bottom surface of the base section;
formed of a pressure-sensitive adhesive material; and
configured to transiently affix the base section to skin of the human patient during the medical procedure.

14. The surgical tracking system of claim 12:
wherein the projection system further comprises:
a mobile cart transiently arranged within the operating space and housing the computer system; and
a housing mounted to the mobile cart;
wherein the optical sensor and the projector are rigidly integrated within the housing;
wherein the optical sensor defines a first field of view facing the region of the body;
wherein the projector defines a second field of view facing the region of the body, the second field of view overlapping the first field of view; and
wherein the projection system defines a composite field of view defined by overlapping regions of the first field of view and the second field of view.

15. The surgical tracking system of claim 12:
wherein the optical sensor comprises an infrared sensor configured to capture
infrared images of the region of the body;
wherein the spherical section of each skin fiducial, in the set of skin fiducials, is formed of the retroreflective material configured to reflect infrared light output by the infrared sensor;
wherein the projection system further comprises a depth sensor configured to capture depth images of the region of the body; and
wherein the computer system is configured to:
detect the set of skin fiducials in an infrared image captured by the infrared sensor;
derive the set of spatial coordinates of the set of skin fiducials on the region of the body relative the projector based on features extracted from the infrared image;
calculate the transform between the three-dimensional medical image and the infrared image by aligning the constellation of skin fiducials in the three-dimensional medical image to the set of spatial coordinates to locate features of the three-dimensional medical image in the operating space;
generate a surface model of the region of the body based on depth data captured by the depth sensor; and
generate the visual overlay based on the set of spatial coordinates, the transform, and the surface model, the visual overlay configured to conform to curvature of the surface of the region of the body during projection onto the region of the body.

16. A method comprising:
accessing an infrared image of a region of a body and captured by an infrared sensor arranged at a first location in an operating space;
detecting a set of skin fiducials arranged in first arrangement on the body in the infrared image, each skin fiducial, in the set of skin fiducials, comprising:
a base section;
an adhesive layer applied to the base section and configured to transiently affix the base section to skin of the body; and
a spherical section coupled to the base section and comprising a retroreflective surface;
for each skin fiducial, in the set of skin fiducials, calculating a set of location coordinates in the operating space based on features extracted from the infrared image;
accessing a three-dimensional medical image of the region of the body, the three-dimensional medical image captured by an intraoperative scanner arranged at a second location in the operating space;
detecting a constellation of skin fiducials in the three-dimensional medical image, the constellation of skin fiducials representing the set of skin fiducials on the body;
calculating a transform between the three-dimensional medical image and the infrared image by aligning the set of skin fiducials in the three-dimensional medical image to the set of location coordinates of the set of skin fiducials in the operating space to locate features of the three-dimensional medical image in the operating space;
accessing a location of a projector in the operating space;
based on the location of the projector, the set of location coordinates, and the transform, generating a visual overlay of the three-dimensional medical image configured for projection onto the region of the body via the projector, the constellation of skin fiducials depicted in the three-dimensional medical image aligned to the set of skin fiducials applied to the region of the body; and
triggering the projector to project the visual overlay onto the region of the body in the operating space.

17. The method of claim 16, further comprising:
receiving confirmation of a medical procedure of a first procedure type for the
human patient;
accessing a first fiducial template, in a set of fiducial templates, corresponding to the first procedure type;
characterizing a difference between the first arrangement and a target arrangement of skin fiducials defined by the first fiducial template;
in response to the difference falling below a threshold difference, verifying the first arrangement for the medical procedure; and
in response to the difference exceeding the threshold difference:
flagging the first arrangement as a setup error;
generating a notification indicating the setup error and comprising a prompt to readjust placement of the set of skin fiducials on skin of the region of the body; and
transmitting the prompt to a medical professional affiliated with the medical procedure.

18. The method of claim 16, wherein accessing the three-dimensional medical image captured by the intraoperative scanner comprises accessing the three-dimensional medical image captured by a CT scanner.

19. The method of claim 16:
further comprising:
accessing a depth image depicting the region of the body and captured by a depth sensor arranged in the operating space; and
defining a three-dimensional mesh representing a surface of the region of the body based on features extracted from the depth image; and
wherein generating the visual overlay of the three-dimensional medical image based on the location of the projector, the set of location coordinates, and the transform comprises generating the visual overlay of the three-dimensional medical image based on the location of the projector, the set of location coordinates, the transform, and the three-dimensional mesh, the visual overlay configured to conform to curvature of the surface of the region of the body during projection onto the region of the body.

20. The method of claim 16:
wherein detecting the set of skin fiducials comprises detecting:
a first skin fiducial transiently affixed to a first location within the region of the body;
a second skin fiducial transiently affixed to a second location within the region of the body; and
a third skin fiducial transiently affixed to a third location within the region of the body; and
wherein calculating the set of location coordinates in the operating space based on features extracted from the infrared image for each skin fiducial, in the set of skin fiducials, comprises:
calculating a first set of location coordinates corresponding to the first location of the first skin fiducial based on a first set of features extracted from the infrared image;
calculating a second set of location coordinates corresponding to the second location of the second skin fiducial based on a second set of features extracted from the infrared image; and
calculating a third set of location coordinates corresponding to the third location of the third skin fiducial based on a third set of features extracted from the infrared image.
